(19) United States
(12) Patent Application Publication
(10) Pub. No.: US 2025/0345590 A1
(43) Pub. Date: Nov. 13, 2025

(54) METHOD AND SYSTEM FOR DETERMINING THE SPEED OF SOUND IN A FLUID IN THE REGION OF A CARDIAC SUPPORT SYSTEM

(71) Applicant: Kardion GmbH, Stuttgart (DE)

(72) Inventors: Thomas Alexander Schlebusch, Renningen (DE); Tobias Schmid, Stuttgart (DE)

(21) Appl. No.: 19/218,142

(22) Filed: May 23, 2025

Related U.S. Application Data
(63) Continuation of application No. 15/734,322, filed on Jun. 14, 2021, now Pat. No. 12,311,160.

(30) Foreign Application Priority Data
Jun. 6, 2018 (DE) 102018208899.3

(51) Int. Cl. A61M 60/546; A61B 8/00; A61B 8/08; A61B 8/12

(52) U.S. Cl. CPC A61M 60/546; A61B 8/0883; A61B 8/12; A61B 8/488; A61B 8/5223; A61M 2205/3375; A61M 2205/702

(57) ABSTRACT

The invention relates to a method for determining the speed of sound in a fluid in the region of an implanted, vascular support system, comprising the following steps: a) sending an ultrasonic signal by means of an ultrasonic sensor b) reflecting the ultrasonic signal on at least one sound reflector, which is visible in the field of vision of the ultrasonic sensor and arranged at a defined distance at least to the ultrasonic sensor or to a further sound reflector, c) receiving the reflected ultrasonic signal, d) determining the speed of sound in the fluid using the reflected ultrasonic signal.

### INCORPORATION BY REFERENCE TO ANY PRIORITY APPLICATIONS

The present application is a continuation of U.S. application Ser. No. 15/734,322 filed on Jun. 14, 2021, which is a National State entry of PCT Patent Application No. PCT/EP2019/064803, filed on Jun. 6, 2019, titled METHOD AND SYSTEM FOR DETERMINING THE SPEED OF SOUND IN A FLUID IN THE REGION OF AN IMPLANTED VASCULAR SUPPORT SYSTEM, which is an International application of and claims the benefit of priority to German Patent Application No. 102018208899.3, filed on Jun. 6, 2018, the contents of each of which are hereby incorporated by reference herein in their entirety as if fully set forth herein for all purposes. Any and all applications for which a foreign or domestic priority claim is identified in the Application Data Sheet as filed with the present application are hereby incorporated by reference under 37 CFR 1.57.

### BACKGROUND OF THE INVENTION

### Field

The invention relates to a method for determining the speed of sound in a fluid in the region of an implanted vascular support system, a system for determining the speed of sound in a fluid in the region of an implanted vascular support system, and an implantable vascular support system. The invention is particularly used in (fully) implanted left heart support systems (LVAD [Left Ventricular Assist Device]).

### Description of the Related Art

Knowledge of the actually circulated blood volume of a heart support system or cardiac support system is medically of great importance, in particular for regulating the (implanted) support system.

Work is therefore being done on integrating ultrasonic-based volume flow measurement technology into the support systems. An ultrasonic Doppler measurement can be used as a measurement method, wherein only a single ultrasonic transducer is required as a transmitter and receiver element, which primarily saves installation space in the implant. The flow velocity can be calculated based on the frequency shift due to the Doppler effect:

Δf=f₀·2v/c·cos(α)

Where Δf is the resulting Doppler frequency shift, f₀ is the frequency of the emitted ultrasound impulse, v is the flow velocity of the medium, c is the speed of sound in the medium, and α is the angle between the ultrasonic sound path and the main flow direction.

In a (heart) support system, v is to be determined, a is generally known, and f₀ is known. The speed of sound c is only approximately known and depends on the composition and properties of the blood. For high measurement quality, it is therefore necessary to explicitly determine the speed of sound c in the blood by measurement.

### SUMMARY

The task of the invention is to specify a method and to provide a system by which the speed of sound in a fluid, in particular the speed of sound of blood in the region of an implanted vascular support system, can be determined.

This object is achieved by the method specified in claim 1 and the system specified in claim 8. Advantageous embodiments of the invention are specified in the dependent claims.

According to claim 1, a method for determining the speed of sound in a fluid in the region of an implanted vascular support system is proposed here, comprising the following steps:
a) Transmitting an ultrasonic signal by means of an ultrasonic sensor,
b) Reflecting the ultrasonic signal on at least one sound reflector, which is arranged in the field of vision of the ultrasonic sensor and at a defined distance, at least to the ultrasonic sensor or to a further sound reflector,
c) receiving the reflected ultrasonic signal,
d) determining the speed of sound in the fluid using the reflected ultrasonic signal.

The vascular support system is preferably a cardiac support system, particularly preferably a ventricular support system. The support system is regularly used to support the circulation of blood in the cardiovascular system of a human, or patient if applicable. The support system can be arranged at least partially in a blood vessel. The blood vessel is, for example, the aorta, in particular in a left heart support system, or the pulmonary trunk (Truncus pulmonalis) into the two pulmonary arteries, in particular in a right heart support system, preferably the aorta. The support system is preferably arranged at the outlet of the left ventricle of the heart or the left ventricle. The support system is particularly preferably arranged in the aortic valve position.

The method is preferably used to measure the speed of sound in blood using ultrasound in a heart support system. The method can contribute toward determining a fluid flow velocity and/or fluid volume flow from a ventricle of a heart, in particular from a (left) ventricle of a heart towards the aorta in the region of a (fully) implanted, (left) ventricular (heart) support system. The fluid is regularly blood. The speed of sound is preferably determined in a fluid flow or fluid volume flow that flows through the support system. The method advantageously makes it possible to also determine the speed of sound or speed of flow required for a (Doppler) measurement in the blood outside of the surgical scenario with high quality, in particular by the implanted support system itself.

The explicit determination of the speed of sound is in particular made possible by integrating one or more sound reflectors in the field of vision of a Doppler ultrasonic sensor of a heart support system, in particular in combination with the enhancement of an additional analysis algorithm, in particular an additional FMCW (frequency-modulated approach)-based analysis algorithm, so that the accuracy of the Doppler-based blood flow measurement is not influenced by uncertainties in the speed of sound. The solution presented here is based in particular on enhancing a vascular support system with an integrated Doppler volume flow sensor with one or more reflectors at a defined distance to the ultrasonic element, so that the speed of sound can be determined based on the geometrically defined and known travel distance between the ultrasonic element and the reflector as well as the measured pulse time of flight and/or beat frequency.

In step a), an ultrasonic signal is emitted by means of an ultrasonic sensor. For this purpose, the ultrasonic sensor preferably comprises an ultrasonic element, which, for example due to its oscillation, is designed to emit one or more ultrasonic signals. A piezo element is particularly preferred for the ultrasonic element. Furthermore, the ultrasonic sensor is preferably aligned such that an angle between the ultrasonic sound path and the main flow direction of the fluid is less than 5°. It is also advantageous if the ultrasonic sensor is designed in the manner of an ultrasonic transducer that is configured both for transmitting and receiving ultrasonic signals, for example in that an ultrasonic element can function as a transmitter and receiver element. The emitted ultrasonic signal can also be referred to as a transmission signal and generally has a specific frequency and/or amplitude. In addition, the transmission signal can also be pulsed or comprise at least an (im-)pulse (for the pulse time of flight approach). Furthermore, the transmission signal can preferably be influenced by frequency modulation, in particular for determining beat frequencies (for the FMCW approach).

In step b), the ultrasonic signal is reflected on at least one sound reflector, which is arranged in the field of vision of the ultrasonic sensor and at a (pre-)defined distance to the ultrasonic sensor and/or to further sound reflector, which is also arranged in the field of vision of the ultrasonic sensor. The field of vision of the ultrasonic sensor is usually determined or formed by its emission characteristic. The sound reflector is preferably arranged circumferentially along an inner circumference of a flow channel of the support system. The at least one sound reflector preferably projects at least partially into a flow path of the fluid or flow channel for the fluid through the support system. This flow path or channel can, for example travel through, or be formed by, a(n) (inlet) cannula. It is particularly preferred in this case that the at least one sound reflector is arranged circumferentially along a(n) (inner) surface of the cannula. This defined distance between the ultrasonic sensor and the sound reflector is preferably in the range of 5 to 35 mm, in particular 5 to 30 mm.

The at least one sound reflector can have at least one air-filled cavity. The at least one sound reflector is preferably oriented and/or aligned such that it causes (only) one reflection or (only) reflections in the direction of the ultrasonic sensor. In other words, the at least one sound reflector is oriented and/or aligned such that it reflects incident ultrasonic waves or signals in particular directly and/or only toward the ultrasonic sensor. Furthermore, the at least one sound reflector is preferably aligned such that a surface of the reflector is oriented in parallel to the incident ultrasonic wavefront. Preferably, the at least one sound reflector is a component of the support system that is separate from the further components (e.g., channel inner wall) that come into contact with the fluid. The at least one sound reflector is preferably mounted or fastened to a channel inner wall of the support system.

In step c), the reflected ultrasonic signal is received. The reflected ultrasonic signal is preferably received by means of the ultrasonic sensor. The received ultrasonic signal can also be referred to as a receiving signal. In particular if several sound reflectors are specified, several reflected ultrasound signals can also be received in step c).

In step d), the speed of sound in the fluid is determined using the reflected ultrasonic signal. For this purpose, the ultrasonic signal can be evaluated or analyzed, for example by means of an analysis unit of the support system, in particular the ultrasonic sensor. A (pulse) time of flight-based approach and/or a so-called FMCW-based approach can be performed in this case.

According to an advantageous embodiment, it is proposed that the ultrasonic signal is reflected on at least two sound reflectors, which are arranged at different distances from the ultrasonic sensor. The two sound reflectors generally have a (pre-)defined distance to each other. This distance is preferably in the range of 1 to 10 mm. By using at least two reflectors at different distances, the accuracy can be advantageously further increased, in particular because uncertainties in the speed of sound of the impedance adjustment layer of the ultrasonic transducer and tissue deposits potentially present thereon can be compensated.

According to an advantageous embodiment, it is proposed that the at least one sound reflector has an acoustic impedance that is greater than the largest acoustic impedance of the fluid or is less than the lowest acoustic impedance of the fluid. The at least one sound reflector preferably has an acoustic impedance that differs by at least 5 MRayl from the acoustic impedance of the fluid. If several sound reflectors are specified, they can have the same acoustic impedance or acoustic impedances that differ from each other. However, all present sound reflectors should have an acoustic impedance that is respectively greater than the largest acoustic impedance of the fluid or less than the lowest acoustic impedance of the fluid. Furthermore, the at least one sound reflector preferably has an acoustic impedance in the range of 2 to 80 MRayl. Furthermore, the at least one sound reflector is preferably formed using one or more of the following materials: Titanium, medical stainless steel, e.g., MP35N, platinum iridium, NiTiNol.

Furthermore, the at least one sound reflector preferably has a reflection factor that is greater than the largest reflection factor of the fluid. A reflection factor of the sound reflector in this case is in particular defined as the reflection factor of the boundary layer between the material of the sound reflector and the fluid. A reflection factor of the fluid is in particular defined as the reflection factor of the boundary layer between blood cells and blood serum. If several sound reflectors are specified, they can have the same reflection factor or reflection factors that differ from each other. However, all present sound reflectors should have a reflection factor that is respectively greater than the largest reflection factor of the fluid. The reflection factor of the at least one sound reflector is preferably in the range of 0.3 to 0.99.

According to an advantageous embodiment, it is proposed that the at least one sound reflector be embedded in an embedding material. The embedding material preferably has an acoustic impedance that essentially corresponds to the acoustic impedance of the fluid. For example, a silicone can be used as the embedding material. Further preferably, the embedding material at least partially, preferably completely, envelops the surface of the sound reflector facing toward the fluid. In particular, the at least one sound reflector (using the embedding material) is preferably embedded in a planar and/or smooth surface. Preferably, the at least one sound reflector (by means of the embedding material) is embedded into a surface, the maximum slope of which is less than the maximum slope of the exterior surface of the sound reflector.

According to an advantageous embodiment, it is proposed that the speed of sound be determined using a (pulse) time of flight-based analysis algorithm. In other words, this means, in particular, that a (pulse) time of flight-based analysis algorithm is used to determine the speed of sound. The pulse-time-based analysis algorithm preferably determines the speed of sound as a function of the defined distance at least between the ultrasonic sensor and the sound reflector or between two sound reflectors and at least one (measured) signal time of flight. Particularly preferably, the signal time(s) of flight is/are determined based on a cross-correlation, in particular between the transmission pulse (pulse of the emitted ultrasonic signal) and the received pulses delayed by the time(s) of flight and reflected on the sound reflectors (pulse of the received, reflected ultrasonic signals).

According to an advantageous embodiment, it is proposed that the speed of sound be determined using an FMCW-based analysis algorithm. In other words, this means in particular that an FMCW-based analysis algorithm is used to determine the speed of sound. FMCW is an acronym for frequency modulated continuous wave.

The FMCW-based analysis algorithm preferably determines the speed of sound as a function of the defined distance at least between the ultrasonic sensor and the sound reflector or between two sound reflectors, a change in a frequency of an ultrasonic signal, and at least one (resulting) beat frequency. Particularly preferably, the speed of sound is determined as a function of the defined distance between the ultrasonic sensor and the sound-reflector and/or between two sound reflectors, the slope of a frequency ramp and at least one (resulting) beat frequency.

Preferably, a beat frequency is determined by and/or for the FMCW-based analysis algorithm. The beat frequency can also be referred to as the differential frequency and/or beat frequency. The beat frequency is advantageously determined from an overlay of the ultrasonic signal (transmitted signal) emitted by the ultrasonic sensor with the reflected ultrasonic signal (receiving signal) received by the ultrasonic sensor. As a rule, the number of beat frequencies determined or to be determined corresponds to the number of (ultra)sonic reflectors. Furthermore, a discrete Fourier transformation (DFT) or fast Fourier transformation (FFT) can preferably be used to determine the beat frequency.

According to a further aspect, a system for determining the speed of sound in a fluid in the region of an implanted vascular support system is proposed, comprising:
an ultrasonic sensor that is arranged in or on the support system,
at least one sound reflector that is arranged in the field of vision of the ultrasonic sensor and at a defined distance at least to the ultrasonic sensor or to a further sound reflector.

According to an advantageous embodiment, it is proposed that at least two sound reflectors be arranged at different distances to the ultrasonic sensor. Furthermore, it is also preferred for the system that the at least one sound reflector be embedded into an embedding material.

According to an advantageous embodiment, it is proposed that an analysis unit is specified in which a pulse-time-based analysis algorithm is stored. Alternatively or cumulatively, an analysis unit can be specified in which an FMCW-based analysis algorithm is stored. The analysis unit is preferably a component of the support system, in particular of the ultrasonic sensor. Furthermore, the analysis unit is preferably configured to execute a method proposed herein. The analysis unit can have a memory in which the pulse time of flight-based analysis algorithm and/or the FMCW-based analysis algorithm is or are stored. In addition, the analysis unit can comprise a microprocessor that can access the memory. The processing unit preferably receives data from an ultrasonic element of the ultrasonic sensor.

According to a further aspect, an implantable vascular support system is proposed, comprising a system proposed herein for determining the speed of sound. The support system is preferably a left ventricular heart support system (LVAD) or a percutaneous, minimally invasive left heart support system. Furthermore, it is preferred that said system can be fully implanted. In other words, this means in particular that the support system is completely in the patient's body and remains there. The support system is particularly preferably configured and/or suitable such that it can be arranged at least partially in a ventricle, preferably the left ventricle of a heart and/or aorta, in particular in an aortic valve position.

Furthermore, the support system preferably comprises a cannula, in particular an inlet cannula and a flow machine, such as a pump. The support system can furthermore comprise an electric motor that is in this case regularly a component of the flow machine. The (inlet) cannula is preferably configured such that it can in the implanted state convey fluid from a (left) ventricle of a heart to the flow machine. The support system is preferably elongated and/or has a hose-like shape. The inlet cannula and the flow machine are preferably arranged in the region of opposite ends of the support system.

The details, features and advantageous embodiments discussed in connection with the method can also occur accordingly in the system and/or the support system presented here and vice versa. In this respect, reference is made in full to the related discussion regarding the detailed characterization of the features.

### BRIEF DESCRIPTION OF THE DRAWINGS

The solution presented herein as well as its technical environment are explained below in more detail based on the figures. It is important to note that the invention is not limited by the shown exemplary embodiments. In particular, unless explicitly stated otherwise, it is also possible to extract partial aspects of the facts explained in the figures, and to combine said partial aspects with other components and/or findings from other figures and/or the present description. The following figures show schematically:

FIG. 1 a sequence of a method presented here in a standard operating sequence,

FIG. 2a a detailed view of an implantable vascular support system,

FIG. 2b a detailed view of a further implantable vascular support system,

FIG. 3 an emission characteristic of an ultrasonic element,

FIG. 4 an illustration of a system presented here,

FIG. 5 an illustration of a pulse time of flight-based approach that can be used here,

FIG. 6 an illustration of an FMCW-based approach that can be used here,

FIG. 7 example graphs of real parts of impedances,

FIG. 8a a detailed view of a system presented here, and

FIG. 8b a detailed view of a further system presented here.

### DETAILED DESCRIPTION

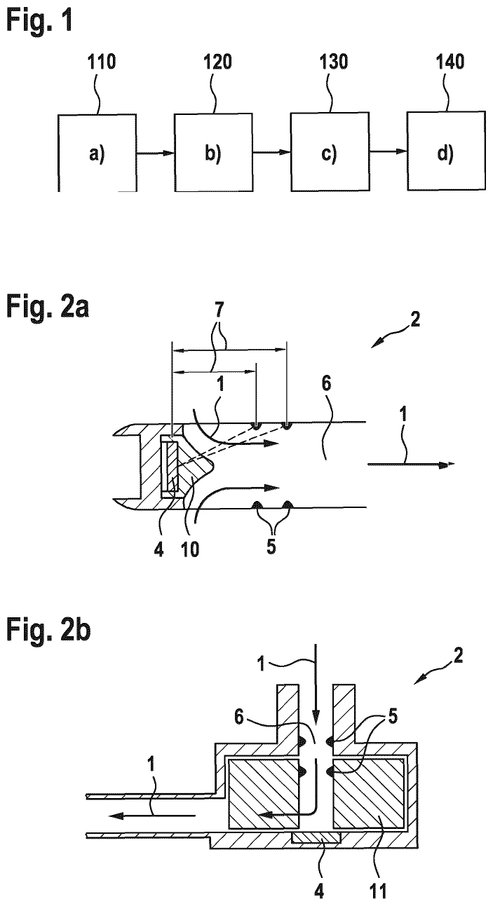

FIG. 1 shows a schematic representation of a sequence of a method presented here in a standard operating sequence. The illustrated sequence of the method steps a), b), c) and d) with the blocks 110, 120, 130 and 140 is only exemplary. In block 110, an ultrasonic signal is transmitted with an ultrasonic sensor. In block 120, the ultrasonic signal is reflected on at least one sound reflector, which is arranged in the field of vision of the ultrasonic sensor and at a defined distance from the ultrasonic sensor. In block 130, the reflected ultrasonic signal is received. In block 140, the speed of sound is determined in the fluid using the reflected ultrasonic signal.

In particular, the method steps a), b), and c) can also be executed at least partially or simultaneously in parallel.

FIG. 2a schematically shows a detailed view of an implantable vascular support system 2. FIG. 2b shows a schematic representation of a detailed view of a further implantable vascular support system 2. FIGS. 2a and 2b are explained jointly below. The reference symbols are used uniformly.

The method presented here can in principle be integrated into all designs of cardiac support systems. By way of example, FIG. 2a shows the integration into a left ventricular microaxial pump in the aortic valve position, and FIG. 2b shows the integration into an apically positioned radial support system 2.

The flow direction of the fluid 1 is represented in FIGS. 2a and 2b by arrows. In each case, an ultrasonic sensor 4 is specified, which is arranged in or on the support system 2. The ultrasonic sensors 4 are designed as an ultrasonic transducer in FIGS. 2a and 2b by way of example. In addition, two circumferential sound reflectors 5 are specified along an inner circumference of a flow channel of the support system 2, which are arranged in the field of vision 6 of the ultrasonic sensor 4 and each at a defined distance 7 to the ultrasonic sensor 4. In particular in the embodiment according to FIG. 2a, the flow channel can be formed in the interior of a(n) (inlet) cannula (not shown here) of the support system 2.

The detailed view according to FIG. 2a shows a tip of a support system 2 with a microaxial pump (not shown here); said tip accommodating the ultrasonic sensor 4. A flow conductive body 10 is in this case by way of example placed directly in front of the ultrasonic sensor 4. Said flow conductive body 10 is not spaced at a distance from the ultrasonic sensor 4 and is permeable for ultrasonic signals. The fluid 1 in this case flows in the direction of the pump. The tip of the support system 2 shown in the detailed view according to FIG. 2a can in a preferred arrangement protrude into a ventricle (not shown here) of a heart with the end shown herein on the left, wherein the pump can be arranged at least partially in the aorta (not shown here). In this arrangement, the support system thus penetrates an aortic valve (not shown here).

The detailed view according to FIG. 2b relates to a support system 2, which is also referred to as an apical radial pump. The support system 2 comprises a flow machine 11 (a pump in this case), which expels the fluid 1 as shown in radial direction.

In both exemplary pump variants, the ultrasonic sensor 4, in particular an ultrasonic element of the ultrasonic sensor 4, is usually placed such that the angle to the flow is α=0° (zero degrees); a best possible Doppler shift can therefore be realized.

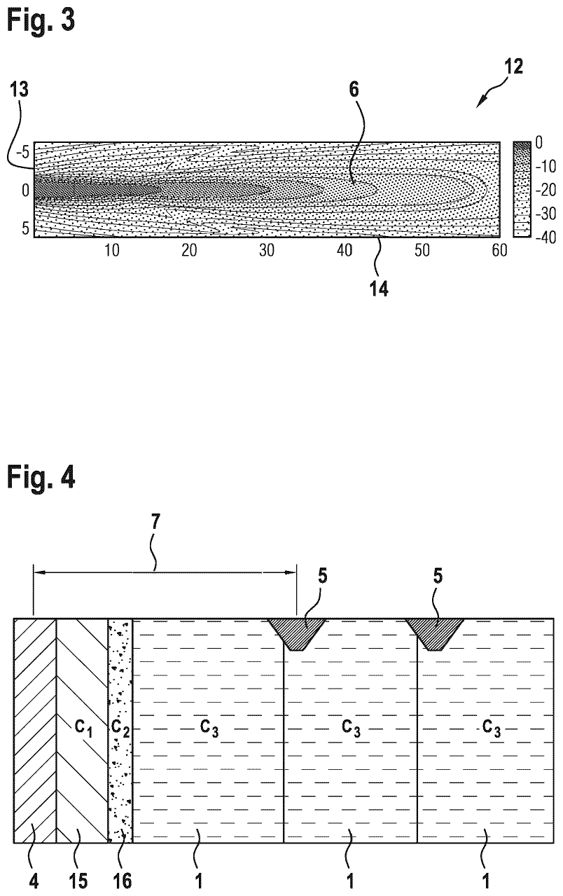

FIG. 3 shows a schematic representation of an emission characteristic 12 of an ultrasonic element (not shown here). The emission characteristic 12 of an ultrasonic sensor or an ultrasonic element of the ultrasonic sensor is generally lobe-shaped with a main beam direction straight ahead. This is shown in FIG. 3 as an example for a circular disk ultrasonic transducer with a diameter of 3 mm at f₀=4 MHz. In other words, FIG. 3 illustrates the field of vision 6 of the ultrasonic sensor (not shown here). A field of vision width 13 can be measured along the ordinate (y-axis) and a field of vision length 14 can be measured along the abscissa (x-axis).

FIG. 4 shows a schematic illustration of a system presented herein. The system comprises an ultrasonic sensor 4 and two sound reflectors 5, which are arranged at a different (defined) distance 7 to the ultrasonic sensor 4. The reflectors 5 project into the fluid 1 by way of example.

Each boundary layer between two acoustic impedances has a reflection factor at which a part of the sound energy is reflected according to the parameter Γ.

$$Γ = \frac{{Z}_{w 2} - {Z}_{w 1}}{{Z}_{w 2} + {Z}_{w 1}} \vert Γ \vert \leq 1$$

In this case, Z_{w1} is the wave impedance before the step point and Z_{w2} is the wave impedance after the step point.

The slightly different acoustic impedance of red blood cells and blood serum, for example, provides the reflected signal, which is usually used to calculate the Doppler frequency shift, from which the flow speed of the blood can be determined.

A(n) (additional) reflector proposed here should preferably have the highest possible reflection factor, which can be achieved in particular by an impedance mismatch with the blood, i.e., the acoustic impedance of the reflector should differ as clearly as possible from the blood, for example by the reflector being made of an air-filled cavity or a metal.

The method with only one reflector 5 can be faulty as soon as more than one unknown medium is present between the ultrasonic sensor 4 and the reflector 5. For example, the acoustic impedance (formula symbol: Z_{W1}) and thus the speed of sound (formula symbol: C₁) of the adjustment layers 15 could change over the years due to water diffusion, or deposits 16 of cell layers (with their own acoustic impedance Z_{w2} and speed of sound C₂) could occur on the ultrasonic sensor 4, thus creating an additional material layer of unknown thickness and/or unknown speed of sound, as shown in greater detail in FIG. 4. In this context, the different speeds of sound of the different media are shown in FIG. 4 by way of example, namely the speed of sound C₁ of the adjustment layers 15, the speed of sound C₂ of the deposits 16 and the speed of sound C₃ of the fluid 1 (here: blood).

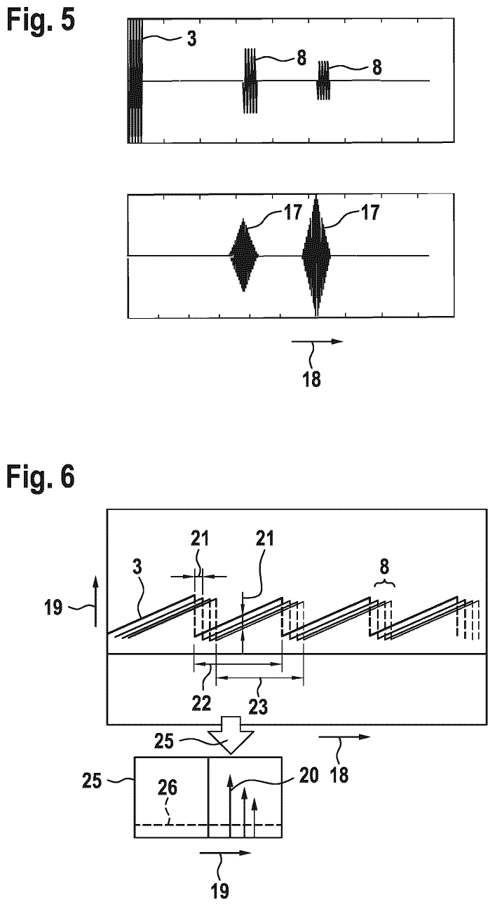

FIG. 5 shows a schematic illustration of a pulse time of flight-based approach usable herein. In order to explain the illustration according to FIG. 5 and/or the pulse time of flight-based approach, reference is also made to the illustration of the system according to FIG. 4.

In addition to the ultrasonic power reflected continuously by each scatter particle of fluid 1 (here: blood; in particular at the respective boundary from blood serum to blood cells), there are clear echoes at the reflectors 5, which can be identified in the received amplitude-time data. In addition, the impulse time of flight from the ultrasonic sensor 4 to the reflector 5 and back to the ultrasonic sensor 4 can be calculated. Since the mechanical design of the (heart) support system 2 and thus the (defined) distance 7 between the ultrasonic sensor 4 and reflector 5 is known, the desired speed of sound c is determined with the formula

$$c = \frac{2 s}{t}$$

where s is the known (defined) distance 7 between the ultrasonic sensor 4 and reflector 5 and t is the measured signal time of flight.

When using two reflectors 5 with different distances 7, as shown in FIG. 4, the time of flight t_{R1} of the impulse scattered on the first reflector 5 is therefore

$${t}_{R 1} = 2 [ \frac{{s}_{1}}{{c}_{1}} + \frac{{s}_{2}}{{c}_{2}} + \frac{{s}_{3}}{{c}_{3}} ]$$

And the time of flight t_{R2} of the pulse scattered on the second reflector 5 is

$${t}_{R 2} = 2 [ \frac{{s}_{1}}{{c}_{1}} + \frac{{s}_{2}}{{c}_{2}} + \frac{{s}_{3}}{{c}_{3}} + \frac{{s}_{4}}{{c}_{3}} ]$$

where s₁ is the thickness of the adjustment layers 15, s₂ is the thickness of the deposits 16, s₃ is the distance between deposits 16 and the first (left) reflector 5 and s₄ is the distance between the first (left) reflector 5 and the second (right) reflector 5, and where c₁ is the speed of sound in the adjustment layers 15, c₂ is the speed of sound in the deposits 16, and c₃ is the speed of sound in the fluid 1 (here: blood).

Since the adjustment layers 15 with the speed of sound c₁ and the deposits 16 with the speed of sound c₂ act equally on both impulses, the difference of the signal times of flight t_{R2}−t_{R1} only contains components in the sought (fluid) range or in the (fluid) range relevant here with the (sought) speed of sound c₃:

$${t}_{R 2} - {t}_{R 1} = 2 [ \frac{{s}_{1}}{{c}_{1}} + \frac{{s}_{2}}{{c}_{2}} + \frac{{s}_{3}}{{c}_{3}} + \frac{{s}_{4}}{{c}_{3}} ] - 2 [ \frac{{s}_{1}}{{c}_{1}} + \frac{{s}_{2}}{{c}_{2}} + \frac{{s}_{3}}{{c}_{3}} ] = 2 \frac{{s}_{4}}{{c}_{3}}$$

Since the distance s₄ of the two reflectors 5 to one another is known, the speed of sound c₃ can be determined independent of the influence of additional layers between the ultrasonic sensor 4 and the reflector 5.

One possibility for determining the times of flight t_{R1} and t_{R2} or t_{R1}−t_{R2} is the calculation of the cross-correlation 17 of the transmission pulse 3 (pulse of the transmitted ultrasonic signal 3) to the receiving pulses 8 (pulses of the received and reflected ultrasonic signals 8) reflected on the ultrasonic reflectors 5 and delayed by the times of flight t_{R1} or t_{R2}. The time-discrete cross correlation 17 can be calculated as follows for an energy signal:

$${R}_{xy} [ n ] = ( x * y ) [ n ] = {\sum }_{m = - ∞}^{∞} {x}^{*} [ m ] y [ m + n ]$$

where R_{xy} [n] is the discrete cross-correlation at time n, and the operator “star” as an acronym for the cross-correlation, x* [m] is the conjugated complex transmission signal over all time shifts m, and y[m+n] is the receiving signal at time n over all time shifts m.

The illustration according to FIG. 5 shows an example of the result of this calculation. FIG. 5 shows the pulse of the emitted ultrasonic signal 3, the pulses of the received reflected ultrasonic signals 8 and the (time-discrete) cross correlation 17 over time 18. The time interval t_{R1}−t_{R2} can be determined from the distance between, e.g., the two tips (peaks) in the cross-correlation signal 17—after reverse-recalculating the discrete time steps.

FIG. 6 shows a schematic illustration of an FMCW-based approach usable here. In order to explain the illustration according to FIG. 6 or the FMCW-based approach, reference is also made to the illustration of the system according to FIG. 4.

The (ultra)sound reflectors 5 represent the dominant targets in the emission range of the ultrasonic sensor 4, in particular due to their high reflection factor. Their beat frequencies can therefore be clearly detected in the calculated spectrum. Since the mechanical design of the (heart) support system and thus the distance between the ultrasonic sensor 4 and the reflector 5 (formula symbol x) is known, the desired speed of sound c is determined by the formula

$$c = 2 * {s}_{x} * \frac{( \frac{bw}{T} )}{{f}_{beat , x}}$$

where sₓ is the known distance between the ultrasonic sensor and reflector x, bw/T is the slope of the frequency ramp, and f_{beat,x} is the resulting beat frequency in the base band. In particular, since the reflectors 5 are installed in a fixed location, the resulting beat frequency is only influenced by their distance to the ultrasonic sensor 4 and the corresponding time of flight of the frequency ramp in the fluid (here: blood), and in particular contains no speed-dependent portion.

When using two reflectors 5 with different distances 7, as shown in FIG. 4, the beat frequency f_{beat,R1} of the frequency ramp reflected at the first reflector is therefore

$${f}_{beat , R 1} = 2 ( \frac{bw}{T} ) [ \frac{{s}_{1}}{{c}_{1}} + \frac{{s}_{2}}{{c}_{2}} + \frac{{s}_{3}}{{c}_{3}} ]$$

and the beat frequency f_{beat,R2} of the frequency reflected on the second reflector is

$${f}_{beat , R 2} = 2 ( \frac{bw}{T} ) [ \frac{{s}_{1}}{{c}_{1}} + \frac{{s}_{2}}{{c}_{2}} + \frac{{s}_{3}}{{c}_{3}} + \frac{{s}_{4}}{{c}_{3}} ]$$

where s₁ is the thickness of the adjustment layers 15, s₂ is the thickness of the deposits 16, s₃ is the distance between deposits 16 and the first (left) reflector 5 and s₄ is the distance between the first (left) reflector 5 and the second (right) reflector 5, and where c₁ is the speed of sound in the adjustment layers 15, c₂ is the speed of sound in the deposits 16, and c₃ is the speed of sound in the fluid 1 (here: blood).

Since the adjustment layers 15 with the speed of sound c₁ and the deposits 16 with the speed of sound c₂ act equally on both frequency ramps, the difference of the beat frequencies f_{beat,R2}−f_{beat,R1} only contains components in the searched (fluid) range or in the (fluid) range relevant here with the (searched) speed of sound c₃:

$${f}_{beat , R 2} - {f}_{beat , R 1} = 2 ( \frac{bw}{T} ) [ \frac{{s}_{1}}{{c}_{1}} + \frac{{s}_{2}}{{c}_{2}} + \frac{{s}_{3}}{{c}_{3}} + \frac{{s}_{4}}{{c}_{3}} ] - 2 ( \frac{bw}{T} ) [ \frac{{s}_{1}}{{c}_{1}} + \frac{{s}_{2}}{{c}_{2}} + \frac{{s}_{3}}{{c}_{3}} ] = 2 ( \frac{bw}{T} ) \frac{{s}_{4}}{{c}_{3}}$$

Since the distance s₄ of the two reflectors 5 to one another is known, the speed of sound c₃ can be determined irrespective of the influence of additional layers between the ultrasonic sensor 4 and the reflector 5.

To determine the beat frequencies, the ultrasonic frequency f₀ is influenced by frequency modulation as an example. Without limitation, sine-wave-shaped, saw-tooth-shaped, triangular or rectangular modulation types can be used. It is particularly preferred that the ultrasonic sensor or the ultrasonic element of the sensor provide a broadband resonance and that the ramp time of flight (formula symbol: T) is much greater than the time of flight of the frequency ramps to the ultrasonic sensor 4 (ultrasound transducer) to the (ultra)-sound reflectors 5 and back again. The echoes of the successively emitted, modulated ultrasonic frequency reflected at the reflectors 5 are overlaid with the instantaneous transmission frequency ramp. The base band signal generated in this way contains the beat frequencies to be determined. These are converted by the transformation into the frequency range, e.g., by discrete Fourier transformation (DFT) or fast Fourier transformation (FFT).

The illustration according to FIG. 6 shows a possible realization of the previously described FMCW-based approach by means of a sawtooth modulation. The upper diagram of FIG. 6 shows the graph of the frequency 19 versus time 18. It can be seen that both the ultrasonic signal 3 (transmission signal) emitted by the ultrasonic sensor and the reflected ultrasonic signals 8 (receiving signals) received by the ultrasonic sensor (three here as an example) are shaped in the manner of a sawtooth. In this case, three receiving signals 8 shifted relative to the transmission signal 3 and to one another are applied as examples, which would for example be the case if three ultrasonic reflectors arranged at different distances to the ultrasonic sensor were used.

The FMCW approach regularly works with a periodic frequency modulation, in this case periodic sawtooth modulation, which should be as time-linear as possible to ensure the best-possible accuracy of the measurement. The modulation is usually performed cyclically. Such a cycle from the lowest to the highest frequency is also referred to as a signal burst. The duration of a corresponding cycle is shown in the upper diagram of FIG. 6 as a so-called chirp duration 22. In addition, a usable chirp duration 23 is marked.

The ultrasonic sensor in this case sends an example of a linear frequency-modulated signal with a sawtooth-shaped change of the transmission frequency 3. The same signal is received by the ultrasonic sensor after a reflection on one of the ultrasonic reflectors. The received signal 8 differs in the time, wherein the time difference 21 between the frequency shifts is generally proportional to the distance of the reflective ultrasonic reflector from the ultrasonic sensor. At the same time (assuming a linear frequency change), the difference frequency 20 between the transmission signal 3 and the receiving signal 8 is the same at any point in time and is thus also a measure for the distance to the reflective ultrasound reflector. This frequency difference can be evaluated in particular in the frequency range.

The frequency plots of the upper diagram in FIG. 6 are in this example used to generate a frequency spectrum 25 by overlaying/multiplying with the instantaneous transmission signal and by means of a subsequent fast Fourier transformation 24, wherein said frequency spectrum 25 carries the difference frequencies 20 in addition to the background noise 26. In a simplified manner, the receiving signal is multiplied with the instantaneous transmission signal, followed by a Fourier transformation of the base band time signal, from which the difference frequencies 20 result, which are also referred to herein as beat frequencies.

The minimum range resolution of FMCW systems is

$$Δ r = \frac{c}{2 * bw} = {s}_{4}$$

is defined. Accordingly, when two ultrasonic reflectors 5 are, e.g., placed at a distance of Δr=s₄=6 mm to each other, and at a(n) (expected) speed of sound in blood c of about 1540 m/s (used to determine the approximately required or particularly advantageous bandwidth), it is possible to work with a bandwidth bw≈128 kHz≤150 kHz.

But a significantly higher range accuracy can be achieved by the additional use of techniques, such as the so-called zero padding (concatenating or padding of zeros) or high-performance frequency estimation methods. This can contribute to a significantly more precise determination of the speed of sound c in the blood. The achievable accuracy depends in particular on the frequency estimation method and/or the signal-to-noise ratio.

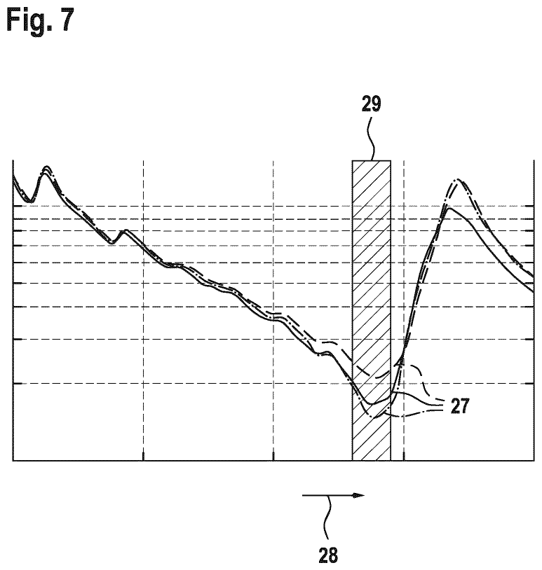

The particularly advantageous linearity can in particular be achieved over the desired frequency band when using piezo elements (as ultrasonic elements), preferably when the quality of the resonance (wide-band resonance) is reduced by backing (amplification). The illustration according to FIG. 7 shows example plots of real components 27 of the impedances of 8 MHz piezo elements versus the stimulation frequency 28. In the case shown, a frequency ramp with the example bandwidth bw=150 kHz could be placed in the frequency band 29 highlighted in gray.

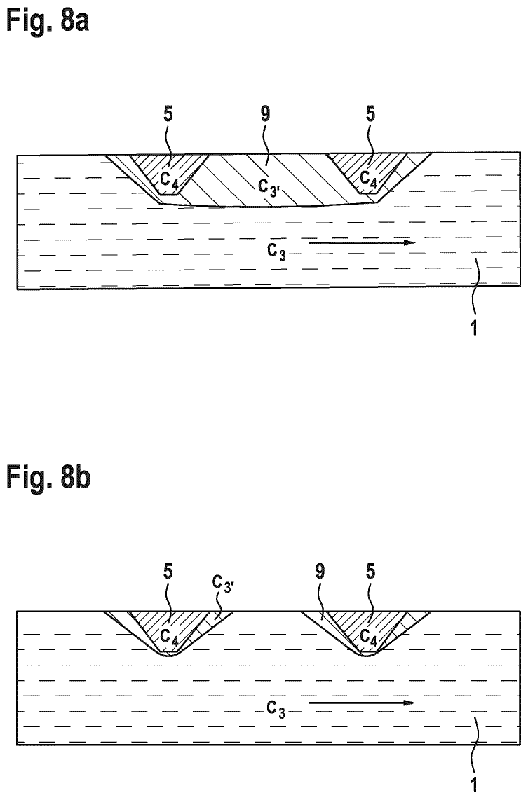

FIG. 8a shows a schematic of a detailed view of a system presented here. FIG. 8b shows a schematic of a detailed view of a further system presented here. FIGS. 8a and 8b are explained jointly below. The reference symbols are used uniformly.

To achieve the best possible reflection, the surface of the reflector should be parallel to the incident ultrasonic wavefront. Since non-planar surfaces such as superimposed reflectors can lead to turbulence in the flow (disadvantageous for Doppler ultrasonic measurement), to the formation of thrombi, and to additional blood damage (hemolysis) due to shear forces, it is expedient to embed the reflectors 5 into an embedding material 9, as illustrated by way of example in FIGS. 8a and 8b. The embedding material 9 is used here as an example for providing a smoother surface or a surface without corners and/or edges in comparison to the reflector surface. It is particularly preferred to embed the at least one reflector 5 into a planar surface, in particular by means of the embedding material 9. The embedding material 9 should as much as possible have the same acoustic impedance as the fluid 1 (here: blood) and be as thin as possible, so that there are no additional reflections or diffractions of the acoustic impulse, unless this additional diffraction is desired. For example, the (or each) reflector 5 with an acoustic impedance C₄ can be embedded into a silicone with an acoustic impedance C_{3′}, wherein C_{3′} is similar to the acoustic impedance C₃ of blood.

The solution presented here in particular has one or more of the following advantages:
By supplementing at least one ultrasonic reflector in the emission range of the ultrasonic system, the speed of sound can be determined from the resulting pulse time of flight and/or the ramp time of flight from the reflector.
The known speed of sound increases the measurement accuracy of the flow measurement.
The speed of sound depends on the composition of the blood and can in this case be determined and used directly.

The FMCW approach does not require a very precise time difference to be measured; an equivalent frequency difference can be determined instead, which significantly reduces the technical effort.

1. (canceled)

2. A method for determining speed of sound in blood within a cannula of a cardiac support system, the method comprising:
transmitting blood through the cannula of the cardiac support system;
sending an ultrasonic signal by an ultrasonic sensor arranged within a flow path of the blood in the cannula of the cardiac support system so that the ultrasonic signal reflects on a first sound reflector and a second sound reflector, the first sound reflector and the second sound reflector being located in a field of vision of the ultrasonic sensor, the first sound reflector and the second sound reflector located at different linear distances from the ultrasonic sensor along the flow path of the blood in the cannula;
receiving a first reflected ultrasound signal corresponding to the ultrasonic signal after reflection at the first sound reflector and a second reflected ultrasonic signal corresponding to the ultrasonic signal after reflection at the second sound reflector; and
determining a speed of sound in the blood based on the first reflected ultrasonic signal, the second reflected ultrasonic signal, and the different linear distances.

3. The method of claim 2, wherein at least one of the first sound reflector and second sound reflector has an acoustic impedance greater than a largest acoustic impedance of the blood or less than a lowest acoustic impedance of the blood.

4. The method of claim 2, wherein at least one of the first sound reflector and the second sound reflector is configured to be embedded into an embedding material.

5. The method of claim 2, wherein the speed of sound is determined based on a pulse time of flight-based analysis algorithm.

6. The method of claim 2, wherein the speed of sound is determined based on a Frequency Modulated Continuous Wave (FMCW) based analysis algorithm.

7. The method of claim 6, wherein a beat frequency is determined.

8. The method of claim 2, wherein at least one of the first sound reflector and the second sound reflector is configured to project sound at least partially into a flow path of the blood formed by an inlet cannula.

9. The system of claim 2, wherein each of the first sound reflector and the second sound reflector is configured to project sound at least partially into a flow path of the blood formed by an inlet cannula.

10. The method of claim 2, wherein the ultrasonic signal is configured to travel along an ultrasonic sound path towards the first sound reflector and second sound reflector and the method further comprises: determining a flow velocity of the blood within a region of the cardiac support system based on a Doppler frequency shift comprising:
$$△ f = {f}_{0} \cdot \frac{2 υ}{c} \cdot cos ( α )$$
wherein Δf is the Doppler frequency shift, f₀ is a frequency of the ultrasonic signal, v is the flow velocity of the blood, c is the speed of sound in the blood, and α is an angle between the ultrasonic sound path and the flow path of the blood.

11. The method of claim 2, wherein the ultrasonic sensor is positioned downstream of the first sound reflector and the second sound reflector.

12. A system for determining speed of sound in blood within a cannula of a cardiac support system, the system comprising:
an ultrasonic sensor arranged in a flow path of blood in the cannula of the cardiac support system; and
at least two sound reflectors, the at least two sound reflectors located in a field of vision of the ultrasonic sensor, wherein the at least two sound reflectors comprise a first sound reflector and a second sound reflector, the first sound reflector and the second sound reflector located at different linear distances from the ultrasonic sensor along the flow path of the blood in the cannula.

13. The system of claim 12, wherein each of the at least two sound reflectors is embedded into an embedding material.

14. The system of claim 12, comprising an analysis unit configured to store a pulse time of flight-based analysis algorithm.

15. The system of claim 12, comprising an analysis unit configured to store a Frequency Modulated Continuous Wave (FMCW) based analysis algorithm.

16. The system of claim 12, wherein each of the at least two sound reflectors is configured to project sound at least partially into a flow path of the blood formed by an inlet cannula.

17. The system of claim 12, wherein the ultrasonic sensor is positioned downstream of the first sound reflector and the second sound reflector.

18. The system of claim 12, wherein at least one of the first sound reflector and the second sound reflector comprises a circumferential sound reflector.

19. A cardiac support system comprising:
an ultrasonic sensor arranged within a flow path of blood in a cannula of the cardiac support system; and
at least two sound reflectors located in a field of vision of the ultrasonic sensor, wherein the at least two sound reflectors comprise a first sound reflector and a second sound reflector, the first sound reflector and the second sound reflector located at different linear distances from the ultrasonic sensor along the flow path of the blood.

20. The system of claim 19, wherein the ultrasonic sensor is positioned downstream of the first sound reflector and the second sound reflector.

21. The system of claim 19, wherein each of the at least two sound reflectors is embedded into an embedding material.
